(19) United States
(12) Patent Application Publication
(10) Pub. No.: US 2004/0254456 A1
(43) Pub. Date: Dec. 16, 2004

(54) Method and apparatus for realistic two-dimensional imaging

(76) Inventor: Dieter Ritter, Furth (DE)

Correspondence Address: SCHIFF HARDIN, LLP, PATENT DEPARTMENT, 6600 SEARS TOWER, CHICAGO, IL, 60606-6473, US

(21) Appl. No.: 10/818,020

(22) Filed: Apr. 5, 2004

(30) Foreign Application Priority Data
Apr. 3, 2003 (DE) 103 15 242.3

(51) Int. Cl. A61B 5/05

(52) U.S. Cl. 600/425

(57) ABSTRACT

In a method and an apparatus for three-dimensional imaging, measurement data, from which 3D images of the inside of the body of an examination subject can be created, are acquired with a 3D imaging apparatus from a region of interest of the examination subject, and from the measurement data a three-dimensional image of the region of interest is reconstructed and shown in at least one view as a section or projection image. Before or during the acquisition of the measurement data, exposures of the exterior region of interest of the examination subject are acquired and are associated (correctly with regard to position) as a textured image with a surface of the region of interest reconstructed from the measurement data. The surface of the region of interest textured in this manner is perspectively represented in the view such that the position of the slice or projection image can be recognized relative to the region of interest. A better association of the represented image with the real examined subject can be achieved, in particular in medical imaging.

### BACKGROUND OF THE INVENTION

1. Field of the Invention

The present invention concerns a method as well as an apparatus for three-dimensional imaging, wherein measurement data, with which 3D images of the inside of the body of an examination subject can be created, are acquired with a 3D imaging apparatus from a region of interest of the examination subject and wherein, from the measurement data, a three-dimensional image of the region of interest is reconstructed and shown in at least one view as a section or projection image.

2. Description of the Prior Art

In known methods for three-dimensional imaging, in particular for medical diagnostics, measurement data of a region of interest of an examination subject, from which a three-dimensional image of the examination subject region can be reconstructed, are acquired by suitable measurement apparatuses, for example magnetic resonance systems, computed tomography systems or C-arm x-ray apparatuses. Newer developments also enable a low-contrast 3D reconstruction of the patient anatomy using a C-arm X-ray apparatus with a motor-controlled C-arm. The advantage of the use of C-arm x-ray apparatuses in comparison with conventional (gantry-type) computed tomography systems is the improved accessibility to the examination subject regions during the image acquisition. C-arm x-ray apparatuses therefore can be flexibly used in an operating room and can generate current image information corresponding to the surgery site situs. This provides image acquisition and representation to the surgeon during the operation. The soft tissue resolution that can be achieved with such an apparatus enables an application, for example in the fields of gastrology, endoscopy, biopsy or brachytherapy. The surgeon can select the usual representation mode for 3D data, MPR, MIP or VRT. However, in these representation modes the viewer has no direct reference of the displayed slice or projection images to the real subject. If the appertaining organs of the patient are not directly in the field of view of the surgeon, without technical support in the viewing of the patient it is difficult for the surgeon to spatially associate the represented slice or projection images.

Different techniques to improve the orientation of the user in such 3D imaging methods or devices are known. One of these techniques uses “augmented reality” in order to produce a reference of the reconstructed image to the actual subject. The position of the represented image thereby can be clearly recognized relative to the patient by means of a positionally correct projection of the 3D image data on the subject, or by mixing the image data into a head-mounted display.

Furthermore, navigation-supported methods are known in which the position of surgical instruments is detected with suitable measurement devices and is shown in real time within the 3D image data set. Using his directed instruments mixed into the image, the surgeon can interactively orient himself or herself.

### SUMMARY OF THE INVENTION

An object of the present invention is to provide a method as well as a device for three-dimensional imaging that offers an improved reference to the examined subject for the viewer of the reconstructed image.

In the inventive method for three-dimensional imaging, measurement data from which 3D images of the inside of the body of an examination subject can be attained are acquired with a 3D imaging apparatus from a region of interest of the examination subject in a known manner, and from the measurement data a three-dimensional image of the region of interest is reconstructed and shown in at least one view as a section or projection image. The 3D imaging apparatus preferably is a tomography apparatus, in particular a C-arm x-ray apparatus. In accordance with the invention, before or during the acquisition of the measurement data, exterior exposures of the region of interest of the examination subject are acquired with at least one camera, such as a video camera, and are associated as a textured image correctly in terms of position with a surface of the region of interest reconstructed from the measurement data. The surface of the region of interest textured in this manner is then shown perspectively in the displayed view such that the position of the section or projection image can be recognized relative to the surface of the region of interest. The perspectively correct representation of the textured surface is calculated from the exposure data, corresponding to the selected view.

In addition to the typical embodiments of the 3D imaging apparatus, the appertaining apparatus has at least one camera that is directed at the examination volume of the imaging apparatus, as well as an evaluation module that undertakes the positionally correct association of the exposures of the camera with the surface of the region of interest reconstructed from the measurement device of the imaging apparatus, provides this reconstructed surface with the associated texture, and shows in perspective the textured surface of the region of interest in the selected view, such that the position of the slice or projection image can be recognized on the monitor relative to the surface of the region of interest.

In addition to the slice or projection image, the user of the inventive method or apparatus thus sees on the monitor, in the view selected by him or her, the surface of the reconstructed region of interest of the examination subject, precisely as it appears to the viewer upon direct viewing of the examination subject. This representation enables the viewer, for example a doctor, to immediately understand the relation between the image data (meaning the selected slice or projection image) and the external patient anatomy. For minimally-invasive intervention, biopsy or plastic surgery, the position of soft tissues relative to the skin surface can be relatively clearly recognized. Access paths through the skin surface thus can be easily identified. An advantage of the present method as well as of the associated apparatus is that the user does not have to wear any additional visualization aid such as, for example, a head-mounted display. As before, the user recognizes the features of interest for him or her on the monitor, but with the additional orientation and association with the examined subject.

The present method can be used in different 3D imaging devices that provide a three-dimensional image of the inside of an examination subject. Examples of such imaging devices are computed tomography systems and magnetic resonance tomography systems. In such devices, however, the camera should be arranged such that it can be moved at least in part around the examination subject in order to enable exposures at different perspectives. The present method can be particularly advantageously used in connection with a harm x-ray apparatus in which the camera is attached to the C-arm. In this manner, during the acquisition of the measurement data by shifting of the C-arm, at the same time the camera moves around the subject to be examined such that automatic exposures are made from different perspectives. The camera is preferably attached in the region of tho x-ray detector, in particular laterally on the image intensifier or on the planar image detector (depending on which detector type is used). A second camera can be attached to the x-ray tube. The cameras, preferably color cameras, are each aligned to the isocenter of the reconstruction volume of the imaging apparatus during the image acquisition.

Knowledge about the position of the camera or cameras relative to the reconstruction volume of the imaging device is necessary for the association of the position of the texture information provided by the camera exposures with the reconstructed surface of the region of interest of the examination subject. The determination of this position can ensue in different manners. Thus, this position can be determined in a C-arm x-ray apparatus by optical evaluation of the x-ray calibration body that is used, for example in a step together with the necessary C-arm calibration. The projection matrices for the camera are determined in a manner analogous to the determination of the image matrices of the x-ray system of the C-arm. In a further embodiment, the position of the camera can be determined once relative to the x-ray detector. An unambiguous association can then be undertaken with knowledge of this position.

### DESCRIPTION OF THE DRAWINGS

FIG. 1 is schematic illustration of an apparatus according to the invention, in the example of a C-arm device

FIG. 2 is a flow chart for implementation of the inventive method

FIG. 3 shows an example of a typical representation of a slice image on the basis of the acquired 3D image data set

FIG. 4 shows an example of the representation according to the inventive method.

### DESCRIPTION OF THE PREFERRED EMBODIMENTS

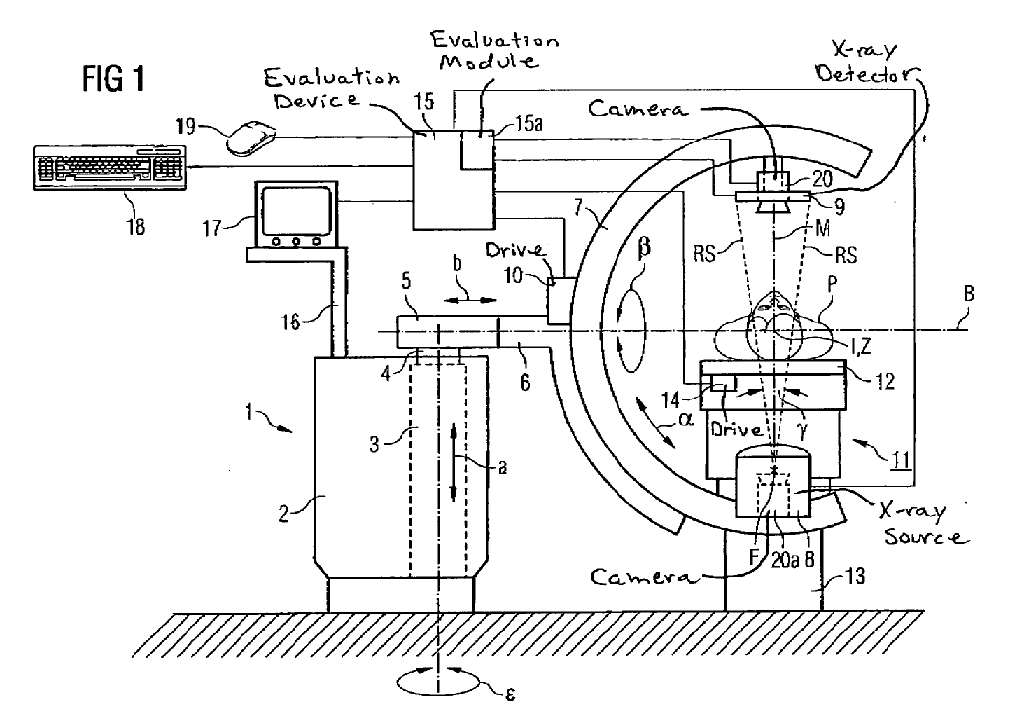

FIG. 1 shows the basic design of a C-arm x-ray apparatus 1 that is fashioned according to the present invention. The apparatus 1 has a base 2 to which is attached (by means of a lifting device 3 indicated schematically in FIG. 1) to a column 4 so as to be rotatable in the directions of the double arrow ε and vertically moveable in the directions of the double arrow. Attached to the column 4 is a holder, to which is attached a bearing part 6 to position a support (the C-arm 7) curved in a C-shape and thus open, which can be moved around an isocenter I.

An x-ray source 8 and a surface-area x-ray detector 9 are attached to the C-arm 7 opposite one another. The x-ray source 8 and the x-ray detector 9 lie opposite one another such that the central ray M (proceeding through the isocenter I) of a conical x-ray beam (originating from a focus F of the x-ray source 8 and indicated in FIG. 1 by its edge rays RS, shown dashed) strikes approximately centrally on the x-ray detector 9. The x-ray detector 9 can be for example, an x-ray image intensifier or, as in the case of the exemplary embodiment, can be as a planar image detector based on a semiconductor panel. The planar image detector has a number of matrix-like detector elements (not shown) arranged, for example, in a detector plane in orthogonal detector columns and rows. The x-ray detector 9 disposed on the C-arm 7 relative to the x-ray source 8 such that, for ideal geometry, the central ray M is at a right angle to the detector plane.

The C-arm 7 is positioned on the bearing part 6 such that it can be moved around the isocenter I (and thus around the system axis Z as a rotation axis of the C-arm 7) in a known manner in the directions of the double arrow α along its circumference by means of a drive device 10 (schematically shown). The system axis Z is perpendicular to the plane of the drawing of FIG. 1, and thus perpendicular to any plane in which the focus of the x-ray source 8 moves given displacement of the C-arm 7 in the α-direction. For this, the drive device 10 includes, for example, an electromotor and a transmission coupling it with the C-arm. By the movement of the x-ray source 8 together with the x-ray detector 9 in the arrow direction α, different first central projections of a subject region to be examined, within which the isocenter I lies, can be acquired. FIG. 1 shows a subject to be examined, for example a patient P, who lies on a positioning device 11. The positioning device 11 has a positioning plate 12 for the patient P that is attached to a base 13 by means of a drive device 14 such that it can be moved in the direction of its longitudinal axis.

The shown 3D imaging apparatus enables a region of interest of the patient P to be scanned by the acquisition of two-dimensional central projections from different projection angles α. A computer 15 as an evaluation device reconstructs from the acquired projections, measurement data representing three-dimensional image information with regard to the scanned volume of the patient P that, for example, can be represented in the form of slice images on a monitor 17 connected to the computer 15. For each projection, a number (corresponding to the number of detector elements of the x-ray detector 9) of measurement values are obtained that provide density information of the portion of the body of the patient P irradiated at this location. Moreover, a keyboard 18 and a mouse 19 that serve for the operation of the device 1 are connected to the computer 15. The computer 15 also is connected to the drive units of the x-ray system as well as to the x-ray source 8 in order to be able to control these components. For acquisition of projections from different projection angles α, the C-arm 7 with the x-ray source 8 and the x-ray detector 9 is moved along its circumference in the direction of the double-arrow α through an angular range that is at least 180° plus the aperture angle Γ of the conical x-ray beam.

Furthermore, the C-arm 7 can be rotated via the bearing part 6 in a known manner around a common axis B (running through the isocenter I and at right angles to the system axis Z) of the holder part 5 and of the bearing part 6 in the directions of the curved double arrow β, and can be positioned on the holding part 5 such that it can be moved in the direction of the axis B according to the double arrow b. The C-arm 7 can rotate in a different plane due to this rotatability around the axis B.

In the present example, a color video camera 20 that is directed toward the isocenter I of the reconstruction volume of the C-arm x-ray apparatus 1 is mounted laterally of the x-ray detector 9. Given the movement of the C-arm in the arrow direction α to acquire the x-ray data, different simultaneous exposures of the exterior of the subject region to be examined can be acquired with the camera 20. The image data provided by the camera 20 are supplied to the evaluation device 15 in the same manner as the measurement data of the x-ray detector 9, and in the evaluation device 15 in the present embodiment an additional evaluation module 15a undertakes the association of the image data representing the surface of the examination subject P as texture information with the surface of the examined region reconstructed from the measurement data of the x-ray detector 9. The image data acquired by the video camera 20 are thereby mixed into this surface by means of the texture mappings known from the graphical data processing. Additionally, for example, a further camera 20a can be arranged on the x-ray source 8 (as this is indicated in FIG. 1) in order to simultaneously acquire exposures from two perspectives.

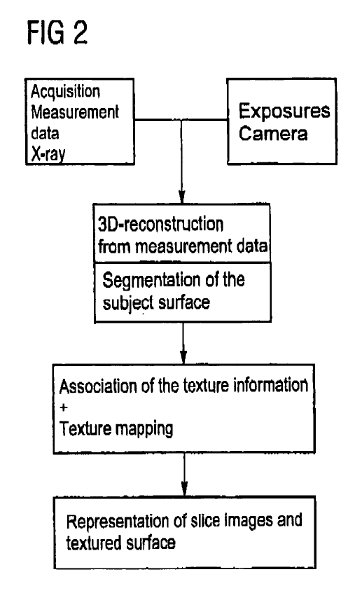

In implementation of the present method, as FIG. 2 schematically shows, the acquisition of the measurement data of the x-ray detector 9 ensues in parallel with the image acquisition with the video camera 20 that is panned around the examination volume with the C-arm 7.

The 3D reconstruction of the examined subject region is subsequently effected from the acquired measurement data, for example by means of the known filtered back-projection method. The subject region is segmented with regard to the background (artifacts, patient beds, etc.), such that the surface of the examined subject region is known in three-dimensional image space. The geometric association of the texture information of the image data acquired by the video camera with this reconstructed subject surface subsequently ensues. After the texture mapping corresponding to this association, the three-dimensional image or the desired three-dimensional image section is shown to the viewer on the monitor together with the desired section or projection image 21 in the selected view, as is, for example visible in FIG. 4. The textured surface 22 of the examined subject region thereby directly accompanies the section or projection section 21.

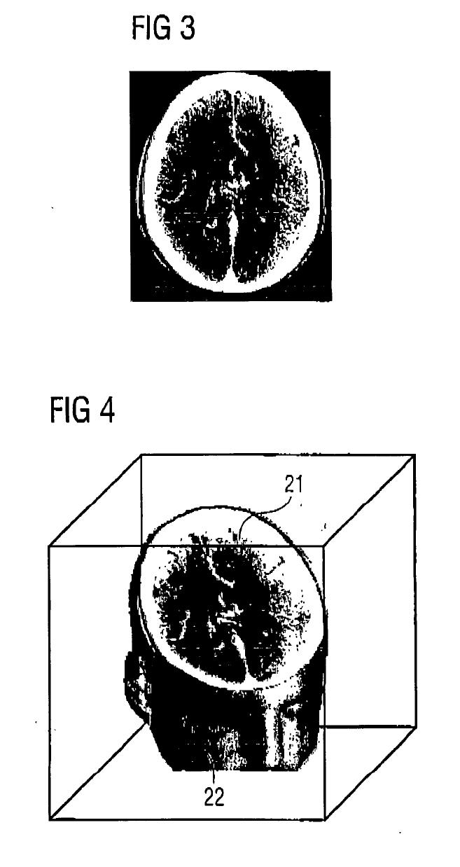

FIG. 3 shows a typical slice image of an examined subject region, in the present case the brain of a patient whereby a slice representation through the brain is recognizable in FIG. 3 (MPR representation). A reference to the patient body lying in front of the surgeon can be deduced by the surgeon from this representation only with difficulty. In comparison to this, FIG. 4 shows a representation as is obtained using the present apparatus or the present method. Here the surface of the head of the patient is additionally provided with the corresponding texture acquired by the video camera and represented correctly with regard to position at the slice image 21 through the brain selected by the doctor. The doctor now sees with a glance precisely the location and the position of the slice image under consideration relative to the patient body. A surgical operation is thereby possible with significantly better orientation in the displayed image.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for three-dimensional imaging comprising the steps of:
acquiring measurement data of an interior of a region of interest of an examination subject;
generating a 3D image of said interior of said region of interest from said measurement data;
before or during acquisition of said measurement data, obtaining an exterior exposure, with a camera, of said region of interest of the examination subject; and
displaying said 3D image and said exterior exposure in combination at a single display with said 3D image displayed as a section or projection image and said exterior exposure being shown as a textured image as a surface of said 3D image for allowing recognition of said slice or projection image relative to said region of interest.

2. A method as claimed in claim 1 comprising obtaining said exterior exposure of said region of interest with a color camera, and displaying said textured image in said display in color.

3. A method as claimed in claim 1 comprising acquiring said measurement data with a C-arm x-ray apparatus by moving a C-arm of the C-arm x-ray apparatus relative to said examination subject, and comprising mounting said camera at said C-arm and moving said camera together with said C-arm to obtain said exterior exposure.

4. A method as claimed in claim 3 wherein said C-arm x-ray apparatus has a radiation detector mounted on said C-arm, and comprising attaching said camera to said radiation detector.

5. A method as claimed in claim 4 wherein the step of obtaining said exterior exposure comprises obtaining a parallity of exterior exposure of the region of interest of the examination subject, and wherein said C-arm x-ray apparatus has an x-ray tube mounted to said C-arm, and comprising attaching a further camera to said x-ray tube and obtaining said parality of exterior exposures respectively with said camera attached to said radiation detector and said further camera attached to said x-ray tube.

6. Am apparatus for three-dimensional imaging comprising:
a data acquisition system for acquiring measurement data of an interior of a region of interest of an examination subject;
a computer for generating a 3D image of said interior of said region of interest from said measurement data;
a camera for, before or during acquisition of said measurement data, obtaining an exterior exposure of said region of interest of the examination subject;
a display device connected to said computer; and
said computer displaying said 3D image and said exterior exposure in combination at said display device with said 3D image displayed as a section or projection image and said exterior exposure being shown as a textured image as a surface of said 3D image for allowing recognition of said slice or projection image relative to said region of interest.

7. An apparatus as claimed in claim 6 wherein said camera is a color camera, and wherein said display device displays said textured image in said display in color.

8. An apparatus as claimed in claim 6 wherein said data acquisition system is a C-arm x-ray apparatus having a C-arm that is moveable relative to said examination subject, and wherein said camera is mounted at said C-arm and moves together with said C-arm to obtain perspective exposure.

9. An apparatus as claimed in claim 8 wherein said C-arm x-ray apparatus has a radiation detector mounted on said C-arm, and wherein said camera is attached to said radiation detector.

10. An apparatus as claimed in claim 9 wherein said C-arm x-ray apparatus has an x-ray tube mounted to said C-arm, and comprising a further camera attached to said x-ray tube and for obtaining a parality of exterior exposures respectively with said camera attached to said radiation detector and said further camera attached to said x-ray tube, as perspective exposures.
